(19) United States
(12) Patent Application Publication
(10) Pub. No.: US 2013/0157319 A1
(43) Pub. Date: Jun. 20, 2013

(54) Method for Simultaneous Fermentation of Pentose and Hexose

(71) Applicant: Feng Chia University, Taichung City (TW)

(72) Inventors: YUN-PENG CHAO, TAICHUNG CITY (TW); CHUNG-JEN CHIANG, TAICHUNG CITY (TW); HONG-MIN LEE, TAICHUNG CITY (TW); ZEI-WEN WANG, TAICHUNG CITY (TW); PO-TING CHEN, TAICHUNG CITY (TW)

(73) Assignee: FENG CHIA UNIVERSITY, TAICHUNG CITY (TW)

(21) Appl. No.: 13/656,654

(22) Filed: Oct. 19, 2012

(30) Foreign Application Priority Data
Dec. 16, 2011 (TW) 100146856

(51) Int. Cl. C12N 15/70; C12P 7/56; C12P 7/06

(52) U.S. Cl. CPC C12N 15/70; C12P 7/065; C12P 7/56
USPC 435/100; 435/471; 435/479; 435/161; 435/139; 435/168; 435/148; 435/167

(57) ABSTRACT

The present invention relates to a method for simultaneous fermentation of pentose and hexose. The present invention modifies the metabolic pathways of a target microorganism in order to enable the target microorganism to rapidly metabolize pentose and hexose at the same time. This present invention simplified the fermentation process, decreased the cost, and increased the efficiency of the fermentation process.

The Sequence Listing ASCII text file, named as “KS-00011-Sequence-Listing.TXT”, sized as “5.41 Kbytes”, and created on Oct. 17, 2012 and submitted on Oct. 19, 2012 in the United States Patent and Trademark Office, is hereby incorporated by reference in this specification. Please attach the above mentioned ASCII text file of Sequence Listing named “KS-00011-Sequence-Listing.TXT” to the end of the specification as a separate part of the disclosure of Sequence Listing in the present application.

The attached ASCII text file of the disclosed “Sequence Listing” will serve as both the paper copy required by 37 C.F.R. §1.821(c) and the computer readable form (CRF) required by 37 C.F.R. §1.821(e). Thus, a statement under 37 C.F.R. §1.821(f) showing that the content of the sequence listing information recorded in the computer readable form is identical to a written copy on paper of “Sequence Listing” is no longer required pursuant to “Legal Framework for EFS-WEB, Section I”.

### BACKGROUND OF THE INVENTION

1. Field of the Invention

The present invention is related to a modified fermentation performance of a microorganism, more particularly to simultaneously utilizing pentose and hexose as the substrates for fermentation.

2. Description of Prior Art

The substitution of renewable resources for the petroleum-related chemicals is the main stream on the international market. Among renewable resources, plant-based biomass (e.g., lignocellulose) is the most abundant in nature. Lignocellulose contains cellulose, hemicellulose, and lignin. After cellulose and hemicellulose are hydrolyzed, the products of hydrolysis are mainly glucose and xylose. In the present invention, Escherichia coli (E. coli) is genetically re-constructed, which is able to metabolize glucose and xylose in a simultaneous and rapid way. The re-constructed E. coli can ferment glucose and xylose and convert them to bio-energy such as alcohol and other chemical such as lactate.

In the existed techniques, E. coli is commonly used to ferment monosaccharides. However, there are several problems to be solved. The advantages of E. coli are rapid growth, easy culturing with a simple medium, easy fermentation operation, and efficient utilization of various monosaccharides. When various monosaccharides are present, E. coli metabolizes glucose first. After glucose is totally consumed, other monosaccharides are utilized. Therefore, E. coli is unable to metabolize different monosaccharides at the same time in the presence of glucose. Therefore, the overall sugar metabolism rate of E. coli is inefficient. In the existing technology, some mutagens such as ultra-violate ray, gamma ray, and nitrosoguanidine are used to mutate bacterial strains. Through the screening process, a strain metabolizing pentose and hexose simultaneously is isolated. However, the mutation method requires repeated screening, which is not systematic and is laborious as well as complicated. The resulting mutant strains are usually inefficient in terms of co-utilization of pentose and hexose.

In FIG. 3, the metabolic pathway of glucose (Glc) and xylose (Xyl) utilization in E. coli is shown. When E. coli metabolizes glucose (Glc), (i.e., hexose), some intermediates can suppress metabolism of other monosaccharides such as xylose (Xyl) (i.e., pentose). Some existed techniques made the phosphotransferase system of glucose deficient, in an attempt to suppress the catabolite repression effect and to increase the uptake rate of other monosaccharides. Although the resulting E. coli could metabolize glucose and pentose simultaneously, the rate of glucose metabolism is decreased significantly. Accordingly, it is not beneficial for the overall fermentation process and lowers the production efficiency.

In the existing technique, two distinct strains able to metabolize glucose and xylose individually are adopted. One strain metabolizes glucose only and the other strain deficient in glucose metabolism utilizes xylose solely. The objective of co-utilization of pentose and hexose is then achieved. However, the process is not easy to operate and the efficiency of the two sugars co-fermentation needs to be optimized by the adjustment of fermentation conditions. In addition, the two strains are cultured, thus increasing the fermentation cost that is unfavorable for industrial applications.

The described drawbacks must be overcome. For example, the cost of fermentation is high, the rate of fermentation is not efficient, and the operation procedure of fermentation is complicated. It is necessary to develop a method to equip the strain with the ability to ferment pentose and xylose simultaneously, which can improve and simplify the procedure of and to increase the efficiency of fermentation. This developed technology is particularly important as long as the issue of production of value-added chemicals from renewable resources is concerned.

### SUMMARY OF THE INVENTION

Bio-industry is a representative of the green industry that is recognized as the fourth industrial revolution. Bio-industry is founded on biotechnology. Comparing to the fossil fuels-based industry, biotechnology can reduce the energy consumption and the environmental pollution. In particular, biotechnology is a technology that can use the renewable resources to achieve the sustainable development and environmental progress. Biomass is the main renewable resources, comprising the wastes from agriculture, forestry, fishing, and animal husbandry and the organic waste released from industry and urban area. Through the process of biorefinery process, the biomass is transformed into the alternative energy for substitution of the petroleum-derived products. The biorefinery industries are growing at a roughly rate of 15% every year, and their market price of total global production will reach 1215 billion US dollars by 2012 (Gobina E, 2007, report code EGY054A, BCC Research publications). Among the renewable resources, lignocellulose is the most abundant and widespread. This biomass for current fermentation studies comes from (1) the agriculture residues from sugar cane residues, straw, chaff, corn straw, (2) non-crop plants such as sword grass, (3) woody plant biomass such as Physic Nut and (4) biowaste such as the residues of vegetable, fruit, pulp, and solid waste from the city (Dietmar P, 2006, Biotechnol J. 1:806-814). In general, lignocellulose comprises of 30-60% cellulose, 20-40% hemicellulose, 10-30% lignin. Cellulose is a polysaccharide in which glucose is linked by β-1,4 glycosidic linkage. Because of the hydrogen bonds between its molecules, they cause the formation of crystallinity and amorphous structure. The hemicellulose is a polysaccharide which is made of pentose and hexose with complicated side branches. The hemicellulose of soft wood is hexose like glucose and the hemicellulose of hard wood is pentose like xylose (Ganapathy S. et al., 2010, Eng. Life Sci. 10:8-18). The cellulose and hemicellulose are hydrolyzed mainly to glucose and xylose. Most microorganisms can metabolize glucose effectively; however, a few microorganisms can ferment xylose poorly. Therefore, the poor use of xylose by microorganisms affects the development of the biorefinery industry.

Comparing to other bacteria, Escherichia coli (E. coli) is a bioprocess-friendly strain. It is characterized as rapid growth, being cultured by simple media formula and easy fermentation operation. Moreover, this bacterium is able to metabolize an array of monosaccharides including pentose (including xylose). However, if there is sufficient glucose in the surrounding, it utilizes glucose first. The metabolism of other monosaccharides is inhibited. After glucose is totally consumed, other monosaccharides will be used sequentially. This slows down the rate of monosaccharide metabolism. Even, it makes the other metabolism uncompleted and ineffective.

Because of aforementioned reasons, the present invention is aimed at metabolic engineering of E. coli. In the step (a) of FIG. 1 and FIG. 2, based on the pathway of glucose and xylose, the ptsG gene sequence encoding a glucose permease in the phosphotransferase system is deleted to reduce the catabolite repression. In the step (b) of FIG. 1 and FIG. 2, the glf gene encoding glucose facilitator from Zymomonas mobilis is introduced to increase the metabolic rate of glucose. In the step (c) of FIG. 1 and step (c) and (d) of FIG. 2, the rpiA, tktA, rpe and talB gene in the pentose phosphate pathway are enhanced by fusion at least one λPRPL promoter with the rpiA, tktA, rpe and talB genes to accelerate the rate of the xylose metabolism in a target microorganism. In the step (d) of FIG. 1 and step (e), (f), (d), and (h), the ldhA, frdA, pta, and poxB genes responsible for the production of organic acids are deleted to reduce the cellular inhibitory effect on the pentose phosphate pathway. In the step (e) of FIG. 1 and step (i) of FIG. 2, the ldhA gene coding for a target product such as lactate is introduced. Except for the ldhA gene, other genes for the synthesis of target products such as alcohol, disaccharide, hydrogen, ketone, alkane, or the combination thereof can also be introduced. Lactate can be produced by the expression of the introduced ldhA gene when the target microorganism ferments glucose and xylose simultaneously. The genetically re-constructed strain (E. coli) is able to metabolize glucose and xylose simultaneously. Moreover, the metabolic rates of glucose and xylose are almost comparable. The processes could be manipulated easily; moreover, the fermentative processes could also be simplified. The abilities of alcohol production and lactate production are illustrated. The techniques develop in the present invention can increase the efficiency of fermentative production, which shows a great potential and promise.

### BRIEF DESCRIPTION OF THE DRAWINGS

FIG. 1 illustrates one of the flowcharts of one embodiment in the present invention.

FIG. 2 illustrates one of the flowcharts of another embodiment in the present invention.

FIG. 3 illustrates the glucose and xylose utilization pathway of Escherichia coli.

FIG. 4 illustrates the DNA electrophoresis gel. Keys: lane 1, the wild-type strain BL21; lane 2, DNA standard marker; lane 3, the strain with the genomic insertion of the anti-kanamycin gene.

FIG. 5 illustrates plasmid pND-glf map. Abbreviations: bla, the anti-ampicillin gene; CI857, the temperature-sensitive CI repressor; lambda PR, λPR promoter; lambda PL, λPL promoter.

FIG. 6 illustrates plasmid pHK-glf map. Abbreviations: km, the anti-kanamycin gene; R6K origin, the origin of R6K replication in E. coli; HK attP, prophage HK attachment site; lambda PR, PR promoter, lambda PL, PL promoter.

FIG. 7 illustrates the DNA electrophoresis gel. Keys: lane 1, DNA standard marker; lane 2, plasmid pHK-glf; lane 3: the strain with the inserted glf gene.

FIG. 8 illustrates plasmid pPhi-80-rTA map. Abbreviations: km, the anti-kanamycin gene; R6K origin, the origin of R6K replication in E. coli; Phi80 attP, prophage 80 attachment site; lambda PR, PR promoter; lambda PL, PL promoter.

FIG. 9 illustrates the DNA electrophoresis gel. Keys: lane 1, DNA standard marker; lane 2, plasmid pPhi80-rTA; lane 3, the strain with the inserted rpe and tktA genes.

FIG. 10 illustrates plasmid pLam-rTB map. Abbreviations: km, the anti-kanamycin gene; R6K origin, the origin of R6K replication in E. coli; lambda attP: prophage attachment site; lambda PR, PR promoter; lambda PL, PL promoter.

FIG. 11 illustrates the DNA electrophoresis gel. Keys: lane 1, DNA standard marker; lane 2, plasmid pLam-rTB; lane 3, the strain with the inserted priA and talB genes.

FIG. 12 illustrates plasmid pMC-poxKm map. Abbreviations: Ap, the anti-ampicillin gene; ColE1 origin, the origin of ColE1 replication in E. coli; poxB-1, the N-terminal region of the poxB gene; poxB-2, the C terminal region of the poxB gene; Km, the anti-kanamycin gene; FRT, the FRT site.

FIG. 13 illustrates the DNA electrophoresis gel. Keys: lane 1, DNA standard marker; lane 2: the poxB gene inserted with the FRT site-flanked anti-kanamycin gene; lane 3: the remaining region of the poxB gene after removal of the anti-kanamycin gene.

FIG. 14 illustrates plasmid pMC-ptaKm map. Abbreviations: Ap, the anti-ampicillin gene; ColE1 origin, the origin of ColE1 replication in E. coli; pta-1, the N terminal region of the pta gene; pta-2, the C terminal region of the pta gene; Km, the anti-kanamycin gene; FRT, the FRT site.

FIG. 15 illustrates the DNA electrophoresis gel. Keys: lane 1: lane 1, DNA standard marker; lane 2: the pta gene inserted with the FRT site-flanked anti-kanamycin gene; lane 3: the remaining region of the pta gene after removal of the anti-kanamycin gene.

FIG. 16 illustrates plasmid pND-pet map. Abbreviations: bla, the anti-ampicillin gene; CI857, the temperature-sensitive CI repressor; lambda PR, αPR promoter; lambda PL, αPL promoter.

FIG. 17 illustrates the sugar consumption profile of recombinant strain BL21/pND-pet and BL-G/pND-pet in the presence of mixed sugars. Symbols: () glucose consumption of strain BL21/pND-pet; (∘) xylose consumption of strain BL21/pND-pet; (▪) glucose consumption of strain BL-G/pND-pet; (□) xylose consumption of strain BL-G/pND-pet.

FIG. 18 illustrates the ethanol production profile of recombinant strain BL21/pND-pet and BL-G/pND-pet in the presence of mixed sugars. Symbols: () strain BL21/pND-pet; (▪) strain BL-G/pND-pet.

FIG. 19 illustrates the sugar consumption profile of recombinant strain BL-Gf/pND-pet and BL21e-RB/pND-pet in the presence of mixed sugars. Symbols: (•) glucose consumption of strain BL-Gf/pND-pet; (∘) xylose consumption of strain BL-Gf/pND-pet; (▪) glucose consumption of strain BL21e-RB/pND-pet ;(□) xylose consumption of strain BL21e-RB/pND-pet.

FIG. 20 illustrates the ethanol production profile of recombinant strain BL-Gf/pND-pet and BL21e-RB/pND-pet in the presence of mixed sugars. Symbols: (•) strain BL-Gf/pND-pet; (▪) strain BL21e-RB/pND-pet.

FIG. 21 illustrates the sugar consumption profile of recombinant strain BL-A4/pND-pet in the presence of mixed sugars. Symbols: (•) glucose consumption; (∘) xylose consumption.

FIG. 22 illustrates the ethanol production profile of recombinant strain BL-A4/pND-pet in the presence of mixed sugars.

FIG. 23 illustrates plasmid pTrc-H/D-Ldh map. Abbreviations: bla, the anti-ampicillin gene; pMB1 ori, the origin of the pMB1 replication in E. coli; lacIQ, the lacI repressor; trc promoter, the trc promoter.

FIG. 24 illustrates the fermentation profile of recombinant strain BL-A4/pTrc-H/D-Ldh in the presence of mixed sugars. Symbols, (∘) glucose consumption; (∇) xylose consumption; ( [symbol] ) lactate production.

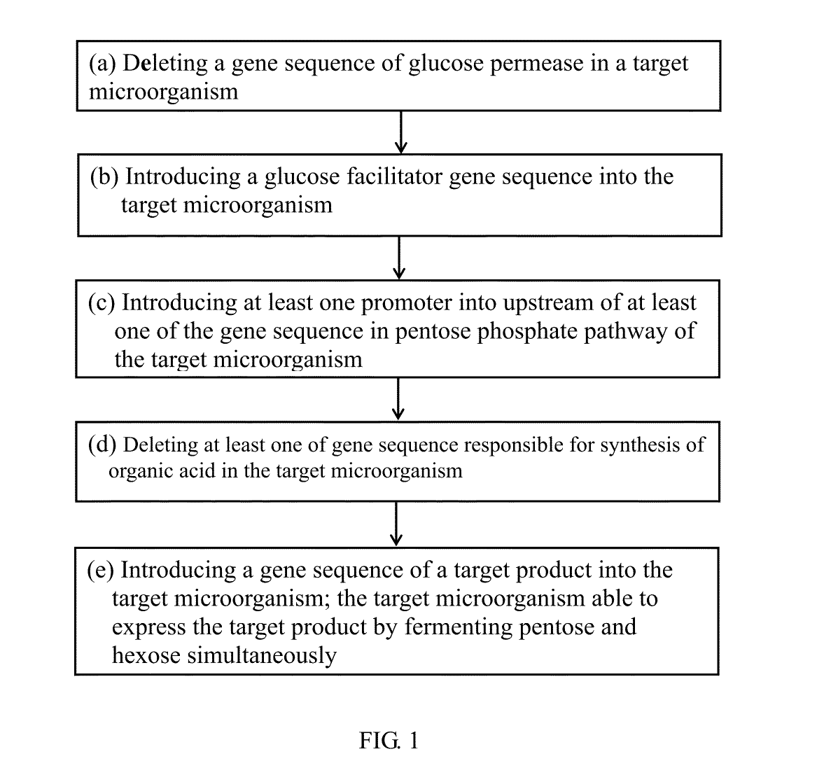

### DETAILED DESCRIPTION ON THE INVENTION

The technologies in the present invention refer to the content in the textbook, such as Sambrook J, Russell D W, 2001, Molecular Cloning: a Laboratory Manual. 3^{rd} ed. Cold Spring Harbor Laboratory Press, New York. The technologies comprise cleavage reaction by restriction enzyme, DNA ligation with T4 ligase, polymerase chain reaction (PCR), agarose gel electropohoresis, sodium dodecyl sulfate-polyacrylamide electrophoresis, and plasmid transformation. All the technologies can be conducted by experienced people who are well acquainted with those. The density of bacteria in the cultured media is measured by spectrophotometer (V530, Hasco) with the wave length at 550 nm; the bacterial density is recorded as OD₅₅₀. The protein assay Reagent (BioRad Co.) is used to measure the concentration of proteins for the total protein quantification. Individually marked protein is analyzed by Alphalmager EP (Alphalnnotech) to quantify the protein resolved by the electrophoresis.

The purification of the chromosome and plasmid of bacteria and phage is carried out by the commercial kit from Wizard® Genomic DNA purification kit (Promega Co.), High-Speed Plasmid Mini Kit (Geneaid Co.) and Gel/PCR DNA Fragments Extraction Kit (Geneaid Co.). The DNA point mutation is carried out by the QuickChange® Sit-Directed Mutagenesis Kit (Stratagene Co.). The restriction enzyme is purchased from New England Boplabs and Fermentas Life Science. The T4 ligase and Pfu DNA polymerase is purchased from the Promega Co. All the primers are synthesized by Mission biotech and Tri-I biotech, Inc. (Taipei, Taiwan).

In the DNA cloning procedure, the bacterial cells used are DH5α (Stratagene Co), BW25142 (Haldimann and Wanner, 2001, J. Bacterior., 183: 6384-93) and BL21 (DE3) (Invitrogen Co.). Bacteria are cultured in LB media (Miller J H, 1972, Experiments in Molecular Genetics, Cold Spring Harbor Laboratory Press, New York). The transformed bacteria are cultured in the media with antibiotics such as: ampicillin (50 μg/mL), kanamycin (50 μg/mL)

The present invention is aimed at developing a process for a microorganism to acquire the ability to simultaneously utilize pentose and hexose as the carbon sources for fermentation. Escherichia coli (E. coli) is used as the main host, because it possesses a lot of advantages and is widely used in industry. Several steps are conducted to achieve the objective. The present invention is detailed by the following descriptions in conjunction with drawings therein.

### Embodiment 1

Deletion of a ptsG Gene Sequence

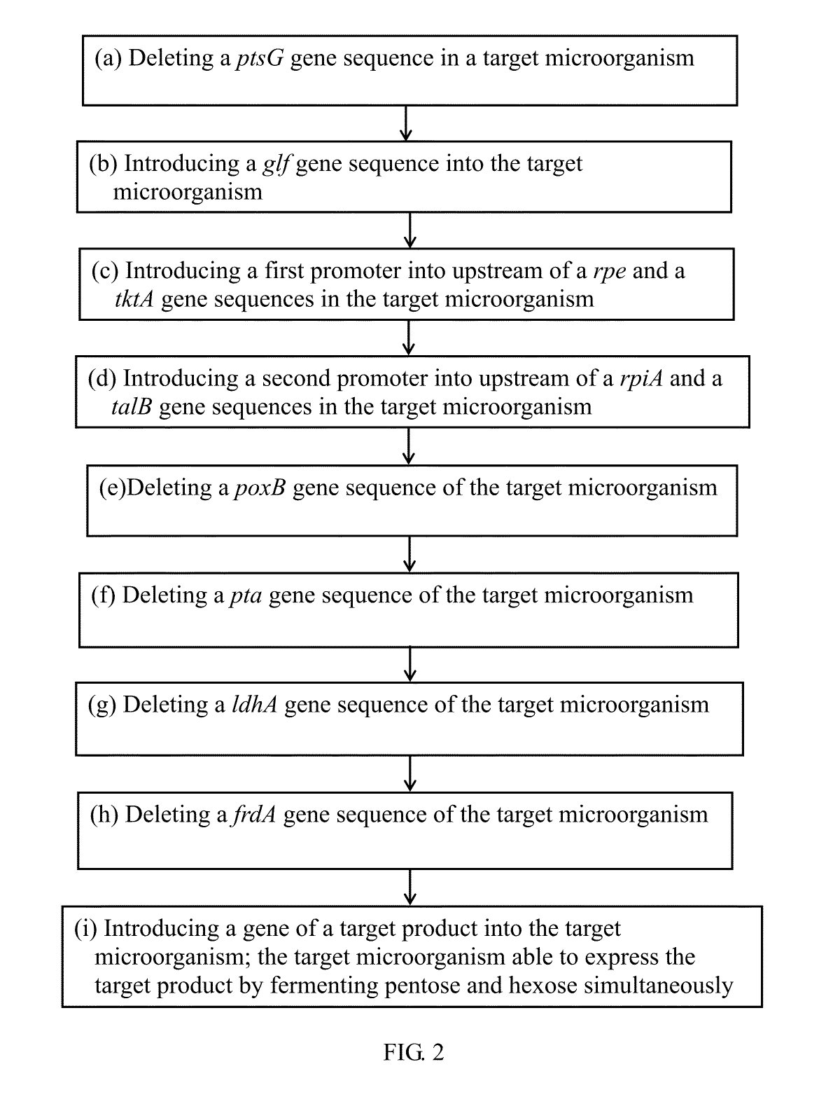

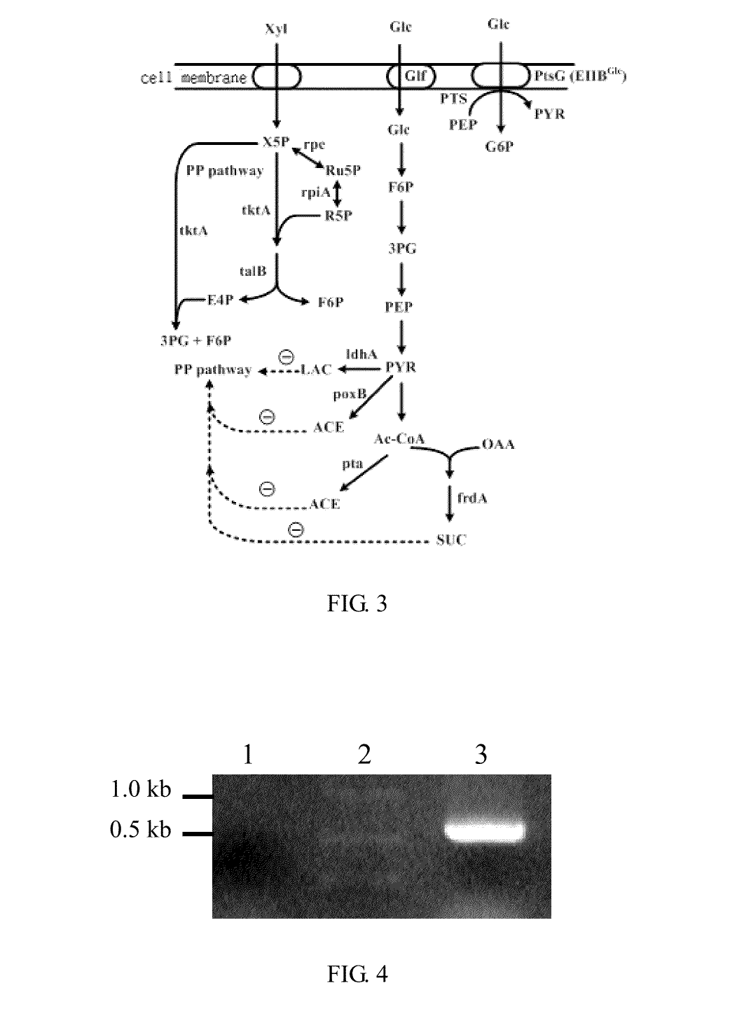

In step (a) of FIG. 2, to reduce a catabolite repression effect, the ptsG gene encoding glucose permease in the phosphotransferase system is deleted from the chromosome of E. coli strain BL21. The purpose of this approach is to make the bacterial strain able to uptake both of xylose and glucose; consequently, to metabolize them. Primer 1 and 2 are synthesized based on the adjacent sequence of the ptsG gene sequence according to the EcoCye database.

Forward primer l
(SEQ ID NO: 1)
(5′-TGGGTGAAACCGGGCTGG)
Reverse primer 2
(SEQ ID NO: 2)
(5′-AGCCGTCTGACCACCACG)
Forward primer 3
(SEQ ID NO: 3)
(5′-GATTGAACAAGATGGATTGC)
Reverse primer 4
(SEQ ID NO: 4)
(5′-GAAGAACTCGTCAAGAAGGC)

The PCR reaction is carried out using the purified chromosome of E. coli strain CGSC 9031(E. coli Genetic Stock Center, USA) as the template and with primer 1 and primer 2. A DNA cassette (2.8 kb) is amplified, and it contained the FRT sites-surrounded anti-kanamycin gene (FRT-kan-FRT) that is flanked by two homologous regions of the ptsG gene sequence. E. coli strain BL21 is transformed with plasmid pKD46 (Datsenko K. A. and Wanner B. L., 2000, Proc. Natl. Aca. Sci. USA, 97:6640-6645) to obtain strain BL21/pKD46. This linear PCR DNA fragment is then transformed into the competent strain BL21/pKD46 by electroporation. The competent cell with linear DNA is cultured in SOC media with 1 mM arabinose at 30° C. to induce the expression of the λ-Red gene sequence on the plasmid. The λ-Red gene sequence product facilitates the homologous recombination between the genomic ptsG gene and the homologous sequences that flank the FRT-kan-FRT of the DNA cassette. After 2-hour incubation, the culture temperature is raised to 42° C. for another 2 hours. Bacterial cells are collected by centrifugation and cultured on LB media with kanamycin. The in situ PCR reaction is carried out with primer 3 and 4 to confirm that bacterial cells carried the inserted copy of the anti-kanamycin gene within the genomic ptsG gene. In FIG. 4, lane 3, the cell colony appearing on LB media with kanamycin is verified to contain the inserted anti-kanamycin gene whereas the anti-kanamycin gene is absent in the wild-type strain BL21 in lane 1 and lane 2 is a DNA marker. To remove the integrated anti-kanamycin gene, plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein whose function is to recombine two FRT sites while leaving a single FRT site behind. Finally, cells that are unable to grow on the LB media with kanamycin are chosen, and one of them is picked up and re-named BL-G.

Construction of a Recombinant E. coli Strain with Introducing the glf Gene
Construction of Integration Plasmid pHK-glf

In the former study, the glucose consumption rate of E. coli strain lacking the ptsG was decreased significantly. Meanwhile, a previous study reported that introduction of the glf gene encoding the glucose facilitator from Zymomonas mobilis (Z. mobilis) could restore the glucose metabolism of E. coli that lost the ability of transporting glucose (Parker C et al., 1995, Mol Microbiol. 15:795-802). In step (b) of FIG. 2, to increase its glucose consumption rate, the glf gene of Z. mobilis is introduced into E. coli strain BL-G with deletion of the ptsG gene.

Forward primer 5
(SEQ ID NO: 5)
(5′-TGTCTCTAGAAGCATGCAGGAGGAATCG)
Reverse primer 6
(SEQ ID NO: 6)
(5′-AGCAACTCGAGTTACTTCTGGGAGCGCCAC)

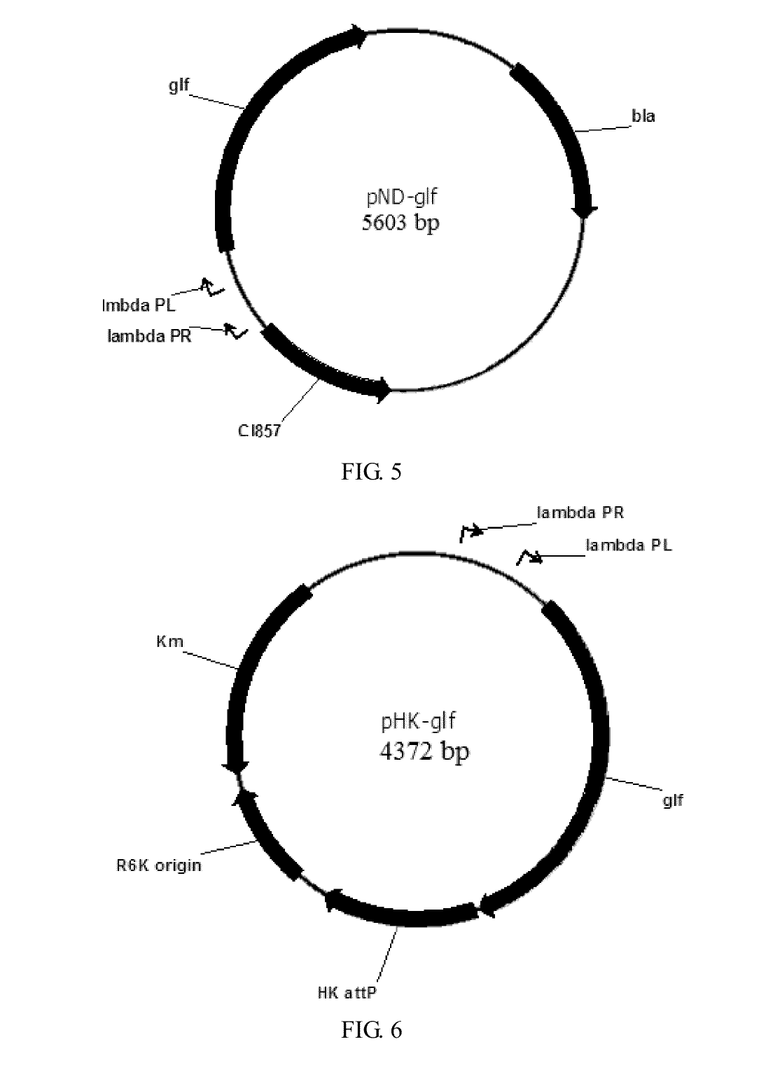

Primers 5 and 6 are synthesized according to the glf gene sequence in the NCBI database. The forward primer 5 contained the XbaI site (underline) while the reverse one carried the XhoI site (underline). The PCR reaction is carried out with aforementioned primers using the Z. mobilis genome as the template. One DNA fragment containing the glf gene sequence is amplified (1.4 kb). After purifying the amplified DNA fragment by Gel/PCR DNA Fragments Extraction Kit, it is cleaved with the restriction enzyme XbaI and XhoI. Plasmid pND707 (Love C A et al., 1996, Gene, 176:49-53) purified by High-Speed Plasmid Mini kit is also cleaved with the XbaI and XhoI. The cleaved DNA fragment is purified and recovered by Gel/PCR DNA Fragments Extraction Kit. T4 ligase is used to incorporate the linearized plasmid pND707 DNA fragment with the glf gene—containing DNA. As a result, plasmid pND-glf is obtained from E. coli strain DH5α as shown in FIG. 5 which illustrates the anti-ampicillin gene (bla), the temperature-sensitive CI repressor (CI857), λPRPL promoter (lambda PR, lambda PL).

Forward primer 7
(SEQ ID NO: 7)
(5′-AAGGGGGATCCATCTAACACCGTGCGTGTTG)
Reverse primer 8
(SEQ ID NO: 8)
(5′-AGCAACTCGAGTTACTTCTGGGAGCGCCAC)

Primers land 8 are synthesized according to the pND-glf; the forward one containing the BamHI site (underline). The PCR is carried out with the primer 7 and primer 8 from plasmid pND-glf. An amplified DNA fragment (1.8 kb) is obtained, and it contained the λPRPL promoter-driven glf gene. The amplified DNA fragment purified by Gel/PCR DNA Fragments Extraction Kit is cleaved with the restriction enzyme BamHI and SmaI. Integration plasmid pHK-Km (Chiang C J et al., 2008, Biotechnol. Bioeng. 101:985-995) purified by High-Speed Plasmid Mini kit is cleaved by BamHI and SmaI. The cleaved DNA fragment is recovered by Gel/PCR DNA Fragments Extraction Kit. The glf gene-containing DNA and the linearized plasmid pHK-Km are spliced together to obtain plasmid pHK-glf from E. coli strain BW25142 as shown in FIG. 6, which illustrates the glf gene (glf), the λPRPL promoter (lambda PR and lambda PL), an anti-kanamycin gene (Km), an origin of R6K replication of E. coli (R6K origin), and phage 80 attachment site (Phi80 attP).

Transformation of Plasmid pHK-glf into Strain BL-G

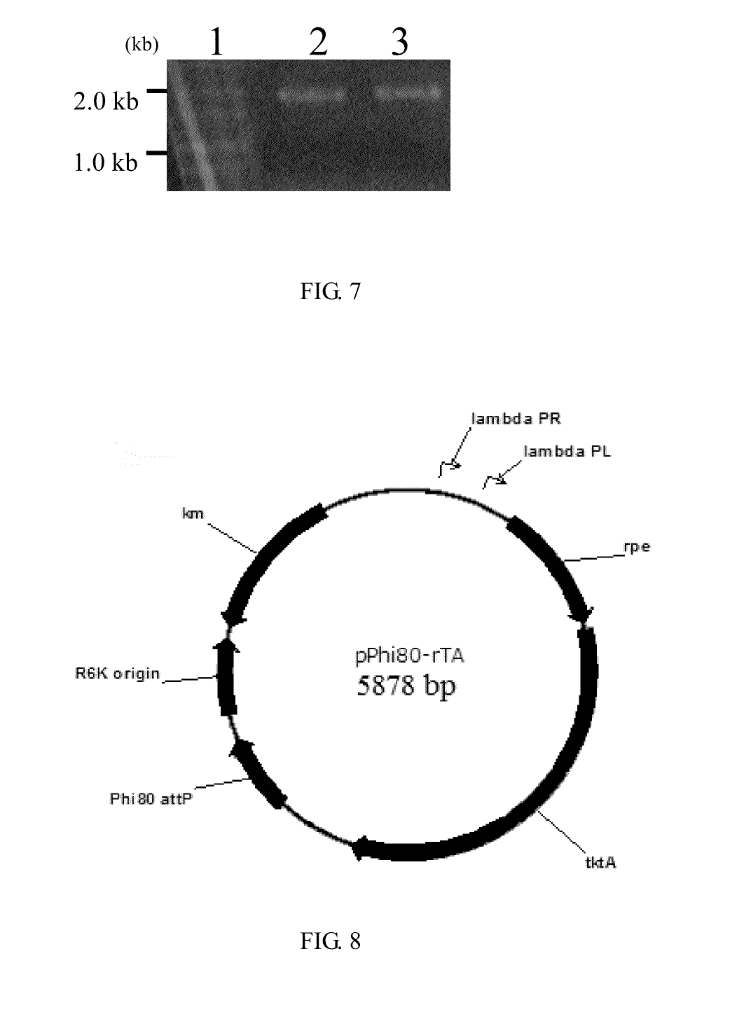

Helper plasmid pAH69 (Haldimann A and Wanner B L., 2001, J Bacteriol., 183:6384-6393) is transformed into strain BL-G by the chemical transformation method to obtain strain BL-G/pAH69. The pHK-glf is then transformed into BL-G/pAH69 to facilitate integration of plasmid pHK-glf. Cells are selected in LB media containing kanamycin and the inserted glf gene is verified by in situ PCR with the primers 7 and 8 as shown in lane 3 of FIG. 7, as compared to lane 1 with the DNA marker and lane 2 with the plasmid pHK-glf. Plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein. The inserted anti-kanamycin gene along with the plasmid backbone is removed by the FLP protein-mediated recombination between two FRT sties. Finally, one of bacterial cells unable to grow on the LB media with kanamycin is chosen and re-named BL-Gf.

Introducing at Least One Gene in the Pentose Phosphate Pathway

The expression of rpe, tktA, rpiA, and talB genes or the combination thereof is enhanced to increase the metabolic rate of xylose in the pentose phosphate pathway. As shown in step (c) and (d) of FIG. 2, the way to increase the expression of the rpe, tktA, rpiA, and talB genes or the combination thereof is to introduce the at least one extra copy of these gene sequence under the control of at least one the λPRPL promoter in the target microorganism.

Enhanced Expression of the rpe and tktA Genes

### Preparing a DNA Fragment Including the rpe Gene

Forward primer 9
(SEQ ID NO: 9)
(5′-TATACATATGAAACAGTATTTGATTGC)
Reverse primer 10
(SEQ ID NO: 10)
(5′-CCTGAATTCAAACTTATTCATGACTTACC)

Primers 9 and 10 are synthesized according to the rpe gene sequence in the database of NCBI; the forward primer containing the NdeI site (underline), the reverse primer containing the EcoRI site (underline). The PCR reaction is carried out with the primers 9 and 10 and the chromosome of BL21 as the template. One DNA fragment (0.7 kb) including the rpe gene is amplified. The amplified DNA fragment is purified by Gel/PCR DNA Fragments Extraction Kit and cleaved with the restriction enzyme NdeI and EcoRI. The cleaved DNA fragment is purified and recovered by the Gel/PCR DNA Fragments Extraction Kit.

Preparing a DNA Fragment Including the tktA Gene

Forward primer 11
(SEQ ID NO: 11)
(5′-ACGGGAATTCAGGAGGAGTCAAAATG)
Reverse primer 12
(SEQ ID NO: 12)
(5′-GGGCCTCGAGTTACAGCAGTTCTTTTC)

Primers 11 and 12 are synthesized according to the tktA gene sequence in the database of NCBI; the forward primer 11 containing the EcoRI site (underline); the reverse primer 12 containing the XhoI site (underline). The PCR reaction is carried out with the primers 11 and 12 and the chromosome of BL21 as the template. One DNA fragment (2.01 kb) including the tktA gene is amplified. The amplified DNA fragment is purified by Gel/PCR DNA Fragments Extraction Kit and cleaved with the restriction enzyme EcoRI and XhoI. The cleaved DNA fragment is purified and recovered by the Gel/PCR DNA Fragments Extraction Kit. Plasmid pND707 (Love C A et al., 1996, Gene, 176:49-53) purified by the High-Speed Plasmid Mini kit is digested with restriction enzyme NdeI and EcoRI and then purified by the Gel/PCR DNA Fragments Extraction Kit. DNA fragments containing the rpe and tktA genes and linearized plasmid pND707 are spliced together to obtain plasmid pND-rTA.

Integration of the rpe and tktA Genes into Strain BL-Gf

Forward primer 13
(SEQ ID NO: 13)
(5′-AAGGGGGATCCATCTAACACCGTGCGTGTTG)
Reverse primer 14
(SEQ ID NO: 14)
(5′-GGGCCTCGAGTTACAGCAGTTCTTTTC)

According to plasmid pND-rTA, the primers 13 and 14 are designed: the forward primer 13 containing the BamHI site (underline). A DNA fragment (2.7 kb) containing the λPRPL promoter-driven rpe and tktA genes is amplified by PCR with the primer 13, 14 and the pND-rTA as the template. The PCR DNA fragment is purified by the Gel/PCR DNA Fragments Extraction Kit and cleaved with the restriction enzyme BamHI. Plasmid pPhi80-km (Chiang C J et al., 2008, Biotechnol. Bioeng. 101:985-995) purified by High-speed Plasmid Mini kit is cleaved by the restriction enzyme BamHI and SmaI. The cleaved fragment is purified by the Gel/PCR DNA Fragments Extraction Kit. The DNA fragment containing the λPRPL promoter-driven rpe and tktA genes and linearized plasmid pPhi80-km are spliced together to obtain plasmid pPhi80-rTA from strain BW25142 as shown in FIG. 8 which illustrates the anti-kanamycin gene (km), the λPRPL promoter (lambda PR and lambda PL), the rpe gene (rpe), the tktA gene (tktA), the origin of R6K replication of E. coli (R6K), and phage 80 attachment site, (Phi80 attP).

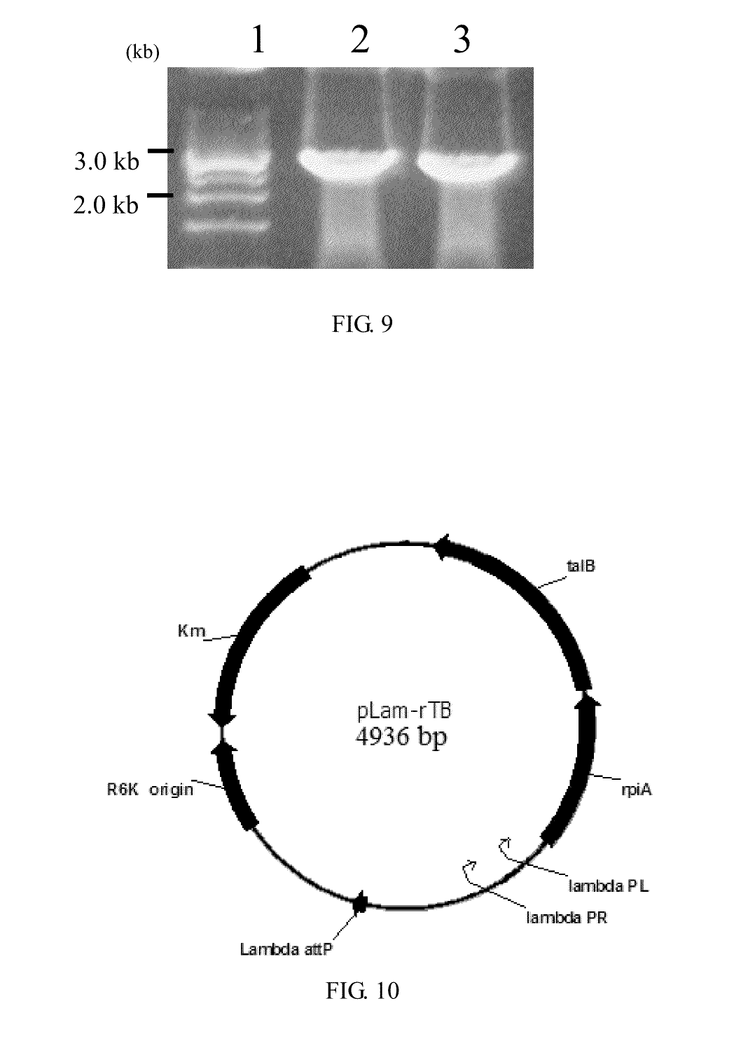

Helper plasmid pAH123 (Haldimann A and Wanner B L., 2001, J Bacteriol., 183:6384-6393) is transformed into strain BL-Gf to obtain strain BL-Gf/pHA123. Followed by transformation of plasmid pPhi80-rTA into the BL-Gf/pHA123, the DNA containing the rpe and tktA genes controlled by the λPRPL promoter is incorporated in to the bacterial chromosome. Cell colonies grown on LB media with kanamycin are picked up and the inserted rpe and tktA genes are verified by in situ PCR based on the primer13 and 14 as shown in lane 3 of FIG. 9 while lane 1 shows the DNA marker and lane 2 shows the plasmid pPhi80-rTA. Plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein. The inserted anti-kanamycin gene sequence along with the plasmid backbone is removed by the FLP protein-mediated recombination between two FRT sties. Finally, one of bacterial cells unable to grow on the LB media with kanamycin is chosen and re-named BL21e.

Enhanced Expression of the rpiA and talB Genes
Preparing a DNA Fragment Including the rpiA Gene

Forward primer 15
(SEQ ID NO: 15)
(5-AATGCCATATGAATTTCATACCACAGGCGAAAC)
Reverse primer 16
(SEQ ID NO: 16)
(5′-TGGAGGAATTCCCGTCAGATCATTTCACAATG)

Primers 15 and16 are synthesized according to the rpiA gene sequence in the database in NCBI; the forward primer 15 containing the NdeI site (underline), the reverse primer 16 containing the EcoRI site (underline). The PCR reaction is carried out with primers 15 and 16 and the chromosome of BL21 as the template. One DNA fragment (0.7 kb) including the rpiA gene is amplified. The amplified DNA fragment is purified by Gel/PCR DNA Fragments Extraction Kit and cleaved with the restriction enzyme NdeI and EcoRI. The cleaved DNA fragment is purified and recovered by the Gel/PCR DNA Fragments Extraction Kit. Preparing a DNA fragment including the talB gene

Forward primer 17
(SEQ ID NO: 17)
(5′-TTTGAATTCAGGAGGATACTATCATGACG)
Reverse primer 18
(SEQ ID NO: 18)
(5′-CTAACTCGAGGTCGACGTTACAGCA GATCGCCGATC 3′)

Primers 17 and 18 are synthesized according to the talB gene sequence in the database in NCBI; the forward primer 17 containing the EcoRI site (underline); the reverse primer 18 containing the XhoI site (underline). The PCR reaction is carried out with the primers 17 and 18 and the chromosome of BL21 as the template. One DNA fragment (1.0 kb) including the talB gene is amplified. The amplified DNA fragment is purified by Gel/PCR DNA Fragments Extraction Kit and cleaved with the restriction enzyme EcoRI and XhoI. The cleaved DNA fragment is purified and recovered by the Gel/PCR DNA Fragments Extraction Kit. Plasmid pND707 (Love C A et al., 1996, Gene, 176:49-53) purified by the High-Speed Plasmid Mini kit is digested with the restriction enzyme NdeI and EcoRI and then purified by the Gel/PCR DNA Fragments Extraction Kit. DNA fragments containing the rpiA and talB genes and linearized plasmid pND707 are spliced together to obtain plasmid pND-rTB.

Integration of the rpiA and talB Genes into Strain BL21e

Forward primer 19
(SEQ ID NO: 19)
(5′-AAGGGGGATCCATCTAACACCGTGCGTGTTG 3′)
Reverse primer 20
(SEQ ID NO: 20)
(5′-CTAACTCGAGGTCGACGTTACAG CAGATCGCCGATC 3′)

According to plasmid pND-rTB, primers 19 and 20 are designed: the reverse primer containing the SalI site (underline). A DNA fragment (1.7 kb) containing the λPRPL promoter-driven rpiA and talB genes is amplified by PCR with the primers 19, 20 and pND-rTB as the template. The PCR DNA fragment is purified by the Gel/PCR DNA Fragments Extraction Kit and cleaved with the restriction enzyme BamHI. Plasmid pLambda-km (Chiang C J et al., 2008, Biotechnol. Bioeng. 101:985-995) purified by High-speed Plasmid Mini kit is cleaved by the restriction enzyme SalI and SmaI. The cleaved fragment is purified by the Gel/PCR DNA Fragments Extraction Kit. The DNA fragment containing the λPRPL promoter-driven rpiA and talB genes and linearized plasmid pLambda-km are spliced together to obtain plasmid pLam-rTB from strain BW25142 as shown in FIG. 10 which illustrates the anti-kanamycin gene sequence (km), the λPRPL promoter (lambda PR, lambda PL), the talB gene sequence (talB), the rpiA gene sequence (rpiA), the origin of R6K replication of E. coli (R6K), the phage λ attachment site (Lambda attP).

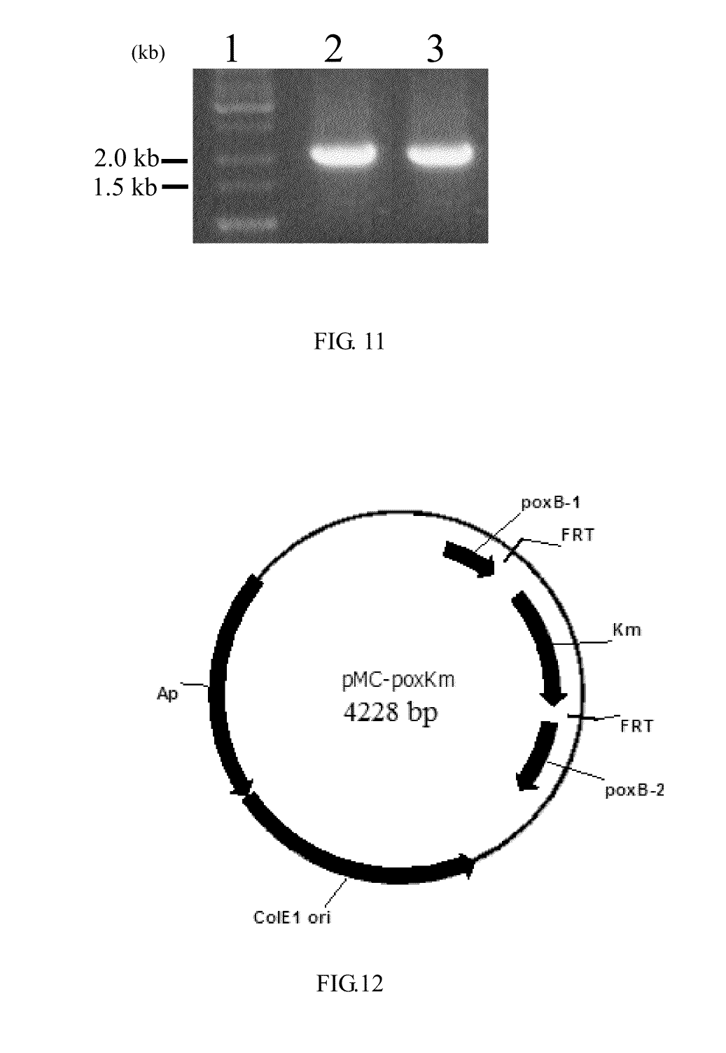

Helper plasmid pAH121 (Haldimann A and Wanner B L., 2001, J Bacteriol., 183:6384-6393) is transformed into strain BL21e to obtain strain BL21e/pHA121. Followed by transformation of plasmid pLam-rTB into the BL21e/pHA121, the DNA containing the rpiA and talB genes controlled by the λPRPL promoter is incorporated in to the bacterial chromosome. Cell colonies grown on LB media with kanamycin are picked up and the inserted rpiA and talB genes are verified by in situ PCR based on primer 19 and 20 as shown as lane 3 of FIG. 11 while lane 1 shows the DNA marker and lane 2 shows the plasmid pLam-rTB. Plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein. The inserted anti-kanamycin gene along with the plasmid backbone is removed by the FLP protein-mediated recombination between two FRT sties. Finally, one of bacterial cells unable to grow on the LB media with kanamycin is chosen and re-named BL21e-RB.

E. coli is able to produce various organic acids under the fermentative condition, known as the mixed acid fermentation. These organic acids are indeed wastes and may exhibit an inhibitory effect on the pentose phosphate pathway. As shown in the step (e), (f), (g), (h) of FIG. 2, at least one ldhA, poxB, pta, frdA gene or the combination thereof responsible for the production of these organic acids is deleted in the target microorganism.

Deletion at Least One Gene Sequence or the Combination Thereof which is Responsible for Synthesis of Organic Acid
Deletion the poxB Gene

Forward primer 21
SEQ ID NO: 21)
(5′-ATTAGAAGCTTGCAGGGGTGAAACGCATCTG)
Reverse primer 22
(SEQ ID NO: 22)
(5′-ATTAGACTAGTGGCTGGGTTGATATCAATC)
Forward primer 23
(SEQ ID NO: 23)
(5′-ATTAGGAATTCGTGATTGCGGTGGCAATC)
Reverse primer 24
(SEQ ID NO: 24)
(5′-ATTAGGTCGACGGTACCAAACTG GCGCAACTGCTG)
Forward primer 25
(SEQ ID NO: 25)
(5′-TTAGGAATTCGTGTAGGCTGGAGCTGCTTC)
Reverse primer 26
(SEQ ID NO: 26)
(5′-ATTCCGGGGATCCGTCGACC)

Primers 21 and 22 are synthesized according to the poxB gene sequence in the database of NCBI; the forward primer 21 containing the HindIII site (underline) and the reverse primer 22 containing the SpeI site (underline). The DNA fragment containing the poxB gene sequence (0.84 kb) is amplified from strain BL21 genome by PCR with the primer 21 and 22. After purifying with the Gel/PCR DNA Fragments Extraction Kit, the PCR DNA is cleaved by the restriction enzyme HindIII and SpeI. The cleaved fragment is recovered by the Gel/PCR DNA Fragments Extraction Kit. Plasmid pMCS-5 (Mo Bi Tec, Germany) purified with the High-speed Plasmid Mini kit is cleaved by HindIII and SpeI and is recovered using the Gel/PCR DNA Fragments Extraction Kit. The poxB gene sequence-containing DNA fragment and linearized plasmid pMCS-5 are ligated together to obtain plasmid pMC-pox from strain DH5α. Primer 23 and 24 are synthesized based on the poxB gene sequence in the database of NCBI; the forward primer 23 containing the EcoRI site (underline) and the reverse primer 24 containing the SalI site (underline). The PCR is carried out with primers 23,-24 and pMC-pox as the template. A DNA fragment (3.5 kb) is amplified. After purification with the Gel/PCR DNA Fragments Extraction Kit, the DNA fragment is cleaved with the restriction enzyme EcoRI and SalI and recovered by the Gel/PCR DNA Fragments Extraction Kit. Moreover, primer 25 and 26 are synthesized according to the sequence of plasmid pKD13 (Datsendo K. A. and Wanner B. L., 2000, Proc. Natl. Aca. Sci. USA, 97:6640-6645) in the database of NCBI ; the forward primer 25 containing the EcoRI site (underline) and the reverse primer 26 containing the SalI site (underline).The PCR is carried out with plasmid pKD13 as the template and with the primers 25 and 26. A DNA fragment (1.3 kb) containing an anti-kanamycin gene sequence flanked by two FRT sites (FRT-kan-FRT) is amplified. After purifying with the Gel/PCR DNA Fragments Extraction Kit, the amplified fragment is cleaved with the restriction enzyme EcoRI and SalI and recovered by the Gel/PCR DNA Fragments Extraction Kit. The FRT-kan-FRT DNA fragment is incorporated into linearized plasmid pMC-pox to obtain plasmid pMC-poxKm as shown in FIG. 12 which illustrates an anti-ampicillin gene sequence (Ap), a origin of ColE1 replication in E. coli (ColE1 ori), a N-terminal region of the poxB gene sequence (poxB-1), a C-terminal region of the poxB gene sequence (poxB-2), the anti-kanamycin gene sequence (Km), and the FRT site (FRT).

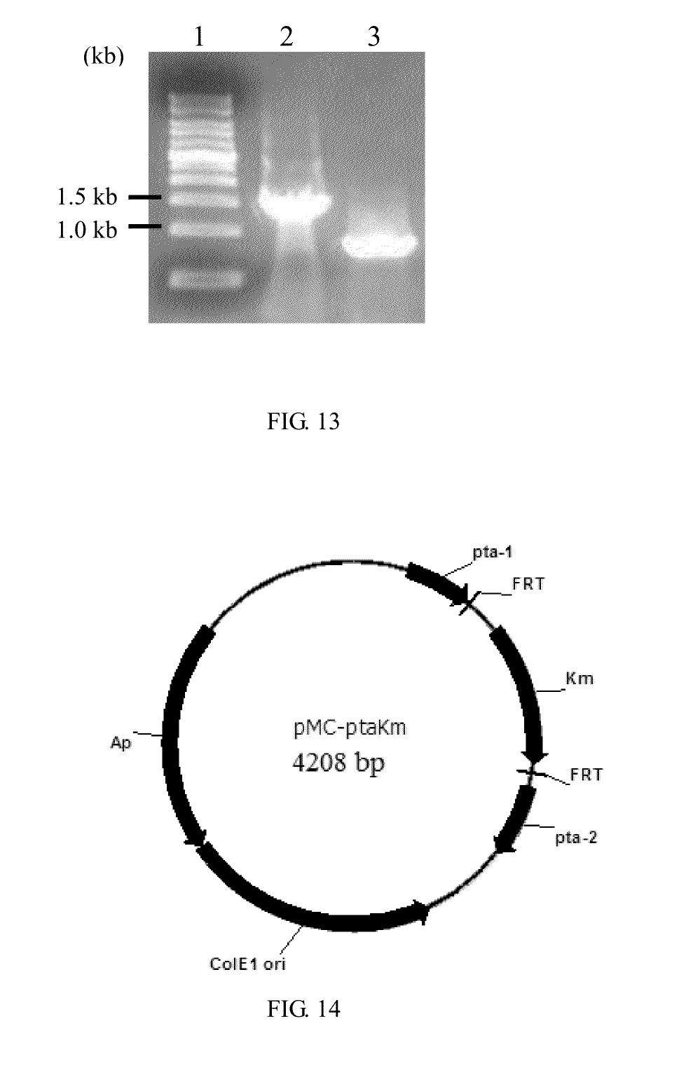

The PCR is carried out with primers 21 and 22 and using plasmid pMC-poxKm as template. The PCR resulted in a DNA cassette (1.9 kb) that contained the FRT-kan-FRT DNA fragment flanked by the homologous regions of the poxB gene sequence, which is purified by the Gel/PCR DNA Fragments Extraction Kit. Helper plasmid pKD46 (Datsenko K. A. and Wanner B. L., 2000, Proc. Natl. Aca. Sci. USA, 97:6640-6645) is transformed into strain BL21e-RB, resulting in strain BL21e-RB/pKD46. The obtained DNA cassette is then transformed into competent strain BL21e-RB/pKD46 by electroporation. The competent cell with linear DNA is cultured in SOC media with 1 mM arabinose at 30° C. to induce the expression of λ-Red gene sequence on the plasmid. The λ-Red gene sequence product facilitates the homologous recombination between the genomic poxB gene sequence and the homologous sequences that flank the FRT-kan-FRT of the DNA cassette. After 2-hour incubation, the culture temperature is raised to 42° C. for another 2 hours. Bacterial cells are collected by centrifugation and cultured on LB media with kanamycin. The in situ PCR reaction is carried out with the primer 21 and 22 to confirm that bacterial cells carried the inserted copy of the anti-kanamycin gene sequence within the genomic poxB gene sequence. To remove the integrated anti-kanamycin gene sequence, plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein whose function is to recombine two FRT sites while leaving a single FRT site behind As depicted in FIG. 13, lane 3 shows the remains of the poxB gene sequence after removal of the anti-kanamycin gene sequence while lane 1 shows the DNA marker and lane 2 shows the poxB gene sequence inserted with the FRT site-flanked the anti-kanamycin gene. Finally, cells that are unable to grow on the LB media with kanamycin are chosen, and one of them is picked up and re-named BL-A1.

### Deletion of the pta Gene

Forward primer 27
(SEQ ID NO: 27)
(5′-TGTCCAAGCTTATTATGCTGATCCCTACC)
Reversed primer 28
(SEQ ID NO: 28)
(5′-GTTCGACTAGTTTAGAAATGCGCGCGTC)
Forward primer 29
(SEQ ID NO: 29)
(5′-ACGATGAATTCCATCAGCACATCTTTCTG)
Reversed primer 30
(SEQ ID NO: 30)
(5′-ACCGTGTCGACGGTACCTGATCGCGACTCGTGC)

Primers 27 and 28 are synthesized according to the pta gene sequence in the database of NCBI; the forward primer 27 containing the HindIII site (underline) and the reverse primer 28 containing the SpeI site (underline). The DNA fragment containing the pta gene sequence (0.95 kb) is amplified from strain BL21 genome by PCR with the primer 27 and 28. After purifying with the Gel/PCR DNA Fragments Extraction Kit, the PCR DNA is cleaved by the restriction enzyme HindIII and SpeI. The cleaved fragment is recovered by the Gel/PCR DNA Fragments Extraction Kit. Plasmid pMCS-5 (Mo Bi Tec, Germany) purified with the High-speed Plasmid Mini kit is cleaved by HindIII and SpeI and is recovered using the Gel/PCR DNA Fragments Extraction Kit. The pta gene sequence-containing DNA fragment and linearized plasmid pMCS-5 are ligated together to obtain plasmid pMC-pta from strain DH5α. Primers 29 and 30 are synthesized based on the pta gene sequence in the database of NCBI; the forward primer containing the EcoRI site (underline) and the reverse primer containing the SalI site (underline). The PCR is carried out with the primers 29, 30 and pMC-pox as the template. A DNA fragment (3.5 kb) is amplified. After purification with the Gel/PCR DNA Fragments Extraction Kit, the DNA fragment is cleaved with the restriction enzyme EcoRI and SalI and recovered by the Gel/PCR DNA Fragments Extraction Kit. Moreover, the primer 25 and 26 are synthesized according to the sequence of plasmid pKD13 (Datsendo K. A. and Wanner B. L., 2000, Proc. Natl. Aca. Sci. USA, 97:6640-6645) in the database of NCBI ; the forward primer 25 containing the EcoRI site (underline) and the reverse primer 26 containing the SalI site (underline).The PCR is carried out with plasmid pKD13 as the template and with the primers 25 and 26. A DNA fragment (1.3 kb) containing an anti-kanamycin gene sequence flanked by two FRT sites (FRT-kan-FRT) is amplified. After purifying with the Gel/PCR DNA Fragments Extraction Kit, the amplified fragment is cleaved with the restriction enzyme EcoRI and SalI and recovered by the Gel/PCR DNA Fragments Extraction Kit. The FRT-kan-FRT DNA fragment is incorporated into linearized plasmid pMC-pta to obtain plasmid pMC-ptaKm as shown in FIG. 14, which illustrates the anti-ampicillin gene sequence (Ap), the origin of ColE1 replication in E. coli, (ColE1 ori), a N-terminal region of the pta gene sequence (pta-1), a C-terminal region of the pta gene sequence (pta-2); the anti-kanamycin gene sequence (Km), and the FRT site (FRT).

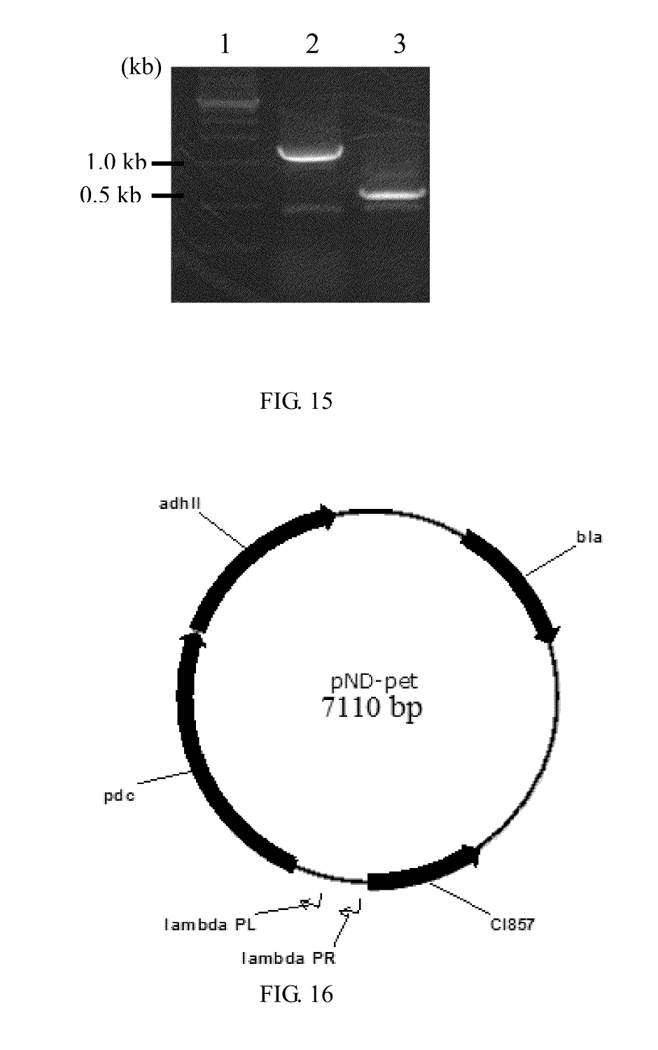

The PCR is carried out with primers 27 and 28 and using plasmid pMC-ptaKm as template. The PCR resulted in a DNA cassette (1.9 kb) that contained the FRT-kan-FRT DNA fragment flanked by the homologous regions of the pta gene sequence, which is purified by the Gel/PCR DNA Fragments Extraction Kit. Helper plasmid pKD46 (Datsenko K. A. and Wanner B. L., 2000, Proc. Natl. Aca. Sci. USA, 97:6640-6645) is transformed into strain BLA1, resulting in strain BLA1/pKD46. The obtained DNA cassette is then transformed into competent strain BLA1/pKD46 by electroporation. The competent cell with linear DNA is cultured in SOC media with 1 mM arabinose at 30° C. to induce the expression of the λ-Red gene sequence on the plasmid. The λ-Red gene sequence product facilitates the homologous recombination between the genomic pta gene sequence and the homologous sequences that flank the FRT-kan-FRT of the DNA cassette. After 2-hour incubation, the culture temperature is raised to 42° C. for another 2 hours. Bacterial cells are collected by centrifugation and cultured on LB media with kanamycin. The in situ PCR reaction is carried out with the primers 27 and 28 to confirm that bacterial cells carried the inserted copy of the anti-kanamycin gene sequence within the genomic pta gene sequence. To remove the integrated anti-kanamycin gene sequence, plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein whose function is to recombine two FRT sites while leaving a single FRT site behind As depicted in FIG. 15, lane 3 shows the remains of the pta gene sequence after removal of the anti-kanamycin gene sequence while lane 1 shows the DNA marker and lane 2 shows the pta gene sequence inserted with the FRT sit-flanked anti-kanamycin gene. Finally, cells that are unable to grow on the LB media with kanamycin are chosen, and one of them is picked up and re-named BL-A2.

Deletion of the ldhA Gene

Forward primer 31
(SEQ ID NO: 31)
(5′-TCTTATGAAACTCGCCGTTTATAG)
Reverse primer 32
(SEQ ID NO: 32)
(5′-TTAAACCAGTTCGTTCGGGCAG)

Primers 3 1 and 32 are synthesized according to the adjacent sequence of the ldhA gene sequence in EcoCye database. The chromosome of CGSC 9216 strain (E. coli Genetic Stock Center, USA) is purified by Wizard Genomic DNA purification kit (Promega Co.). With the primers 31 and 32, the PCR is conducted using the purified chromosome of CGSC 9216 as the template. A DNA cassette (2.8 kb) comprising the FRT site-surrounded anti-kanamycin gene sequence (FRT-kan-FRT) that is flanked by two homologous regions of the ldhA gene sequence is amplified and then purified by the Gel/PCR DNA Fragments Extraction Kit. Helper plasmid pKD46 (Datsenko K. A. and Wanner B. L., 2000, Proc. Natl. Aca. Sci. USA, 97:6640-6645) is transformed into strain BL-A2 to obtain strain BL-A2/pKD46. This linear PCR DNA fragment is then transformed into the competent strain BL-A2/pKD46 by electroporation. The competent cell with linear DNA is cultured in SOC media with 1 mM arabinose at 30° C. to induce the expression of the β-Red gene sequence on the plasmid. The β-Red gene sequence product facilitates the homologous recombination between the genomic ldhA gene sequence and the homologous sequences that flank the FRT-kan-FRT of the DNA cassette. After 2-hour incubation, the culture temperature is raised to 42° C. for another 2 hours. Bacterial cells are collected by centrifugation and cultured on LB media with kanamycin. The in situ PCR reaction is carried out with the primers 31 and 32 to confirm that bacterial cells carried the inserted copy of the anti-kanamycin gene sequence within the genomic ldhA gene sequence. To remove the integrated anti-kanamycin gene sequence, plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein whose function is to recombine two FRT sites while leaving a single FRT site behind Finally, cells that are unable to grow on the LB media with kanamycin are chosen, and one of them is picked up and re-named BL-A3.

Deletion of the frdA Gene

Forward primer 33
(SEQ ID NO: 33)
(5′-GAAAGTCGACGAATCCCGCCCAGG)
Reverse primer 34
(SEQ ID NO: 34)
(5′-CAAGAAAGCTTGTTGATAAGAAAGG)

Primers 33 and 34 are synthesized according to the adjacent sequence of the frdA gene sequence in EcoCye database. The chromosome of CGSC 10964 strain (E. coli Genetic Stock Center, USA) is purified by Wizard Genomic DNA purification kit (Promega Co.). With the primers 33 and 34, the PCR is conducted using the purified chromosome of CGSC 9216 as the template. A DNA cassette (3.0 kb) comprising the FRT site-surrounded anti-kanamycin gene sequence (FRT-kan-FRT) that is flanked by two homologous regions of the frdA gene sequence is amplified and then purified by the Gel/PCR DNA Fragments Extraction Kit. Helper plasmid pKD46 (Datsenko K. A. and Wanner B. L., 2000, Proc. Natl. Aca. Sci. USA, 97:6640-6645) is transformed into strain BL-A3 to obtain strain BL-A3/pKD46. This linear PCR DNA fragment is then transformed into the competent strain BL-A3/pKD46 by electroporation. The competent cell with linear DNA is cultured in SOC media with 1 mM arabinose at 30° C. to induce the expression of the λ-Red gene sequence on the plasmid. The λ-Red sequence gene product facilitates the homologous recombination between the genomic frdA gene sequence and the homologous sequences that flank the FRT-kan-FRT of the DNA cassette. After 2-hour incubation, the culture temperature is raised to 42° C. for another 2 hours. Bacterial cells are collected by centrifugation and on cultured on the LB media with kanamycin. The in situ PCR reaction is carried out with the primers 33 and 34 to confirm that bacterial cells carried the inserted copy of the anti-kanamycin gene sequence within the genomic frdA gene sequence. To remove the integrated anti-kanamycin gene sequence, plasmid pCP20 (Datsenko K. A. and Wanner B. L., 2000, Proc. Natl. Aca. Sci. USA, 97:6640-6645) is transformed into the bacterial strains and induced by shifting the culture temperature from 30° C. to 40° C. to express the FLP protein whose function is to recombine two FRT sites while leaving a single FRT site behind Finally, cells that are unable to grow on the LB media with kanamycin are chosen, and one of them is picked up and re-named BL-A4.

### Embodiment 2

Production of Ethanol in the Constructed Strain by Fermentation of Glucose and Xylose

Construction of Plasmid pND-Pet

The pdc gene encoding pyruvate decarboxylase and the adhII gene encoding alcohol dehydrogenas from Z. mobilis have been studied previously (Ingram Lo et al., 1987, Appl. Environ. Microbiol. 53:2420-2425). The two genes mediate a two-step reaction by conversion of pyruvate to ethanol. In the step (i) of FIG. 2, to enhance ethanol production in E. coli, the pdc and adhII genes are introduced into the genetically constructed E. coli strains as detailed in the following.

Forward primer 35
(SEQ ID NO: 35)
(5′-TATACATATGAGTTATACTGTCGGTAC)
Reverse primer 36
(SEQ ID NO: 36)
(5′-CCATGGATCCTTATCCTCCTCCGAGGAGCTTG)
Forward primer 37
(SEQ ID NO: 37)
(5′-ATGTGGATCCAGGATATAGCTATGGCTTCTTCAACTTTTTATATTC)
Reverse primer 38
(SEQ ID NO: 38)
(5′-AGGACTCGAGTTAGAAAGCGCTCAGGAAGAG)

Primers 35 and 36 are synthesized according to the pdc gene sequence in NCBI database; the forward primer 35 containing the NdeI site (underline) and the reverse primer 36 containing the BamHI site (underline). With the primers 35 and 36, the PCR is carried out using the chromosome of Z. mobilis as the template. A DNA fragment (1.7 kb) containing the pdc gene is amplified and purified by the Gel/PCR DNA Fragments Extraction Kit. Followed by digestion with BamHI and NdeI, the pdc gene-containing DNA fragment is purified by the Gel/PCR DNA Fragments Extraction Kit. Primers 37 and 38 are synthesized according to the adhII gene sequence in NCBI database; the forward primer 37 containing the BamHI site (underline) and the reverse primer 38 containing the XhoI site (underline). With the primers 37 and 38, the PCR is carried out using the chromosome of Z. mobilis as the template. A DNA fragment (1.15 kb) containing the adhII gene is amplified and then purified by the Gel/PCR DNA Fragments Extraction Kit. Followed by digestion with BamHI and XhoI, the adhII gene-containing DNA fragment is recovered by the Gel/PCR DNA Fragments Extraction Kit. Plasmid pND707 purified with the High-Speed Plasmid Mini kit is cleaved by restriction enzyme NdeI and XhoI and followed by purification with the Gel/PCR DNA Fragments Extraction Kit. The linearized plasmid pND707 and the DNA fragments containing the pdc and adhII genes are spliced together to obtain plasmid pND-pet from E. coli strain DH5α as shown in FIG. 16, which illustrates the pdc gene (pdc) and the adhII gene (adh II) driven by the λPRPL promoter (lambda PR and lambda PL), the anti-ampicillin gene (bla), and the temperature-sensitive CI repressor (CI857).

Finally, plasmid pND-pet is transformed into wild-type strain BL21 and genetically constructed strain BL-G, BL-Gf, BL21e-RB and BL-A4 to obtain recombinant strains BL21/pND-pet, BL-G/pND-pet, BL-Gf/pND-pet, BL21e-RB/pND-pet, and BL-A4/pND-pet, respectively.

The fermentation performance of the 5 recombinant strains is investigated by determining the ethanol production and the sugar consumption rate in the presence of mixed sugars (i.e., glucose and xylose). The results are shown as follows:

A single colony of each recombinant strain is picked up and cultured in the 5 mL LB broth with ampicillin at 30° C. and 200 rpm overnight. Each of described strains is seeded respectively in the 25 mL fresh LB broth with ampicillin plus 3% glucose and 3% xylose. The initial optical density (550 nm) of cells reached 2.0. The cell culture is then carried out at 37° C. and 150 rpm. The concentration of glucose, xylose, and ethanol are measured along the time course.

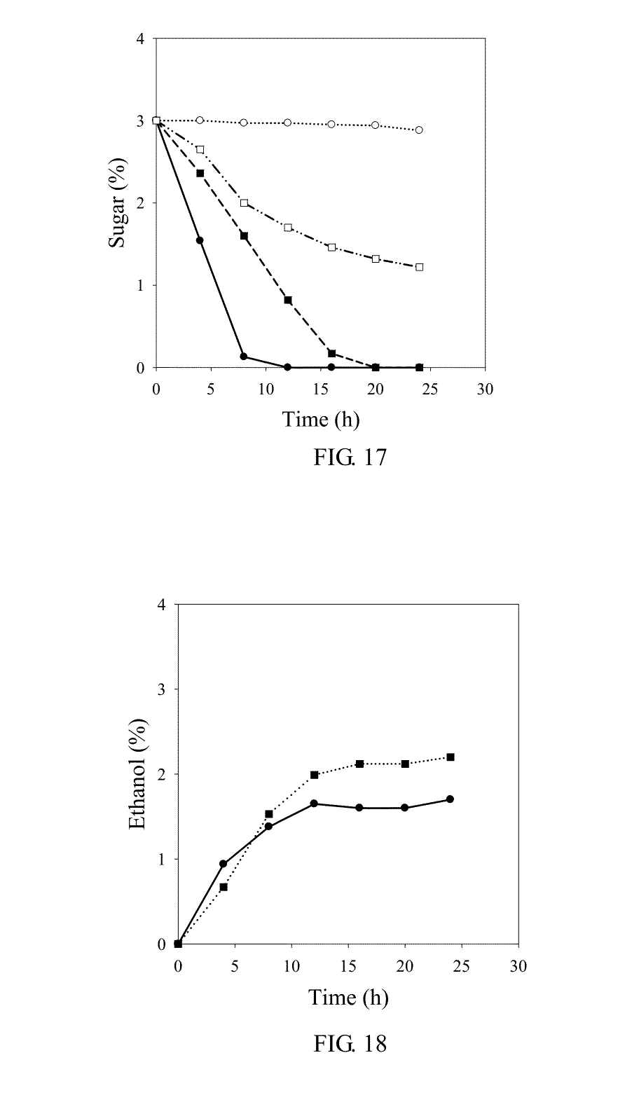

In FIG. 17, the consumption of glucose and xylose for strain BL21/pND-pet and BL-G/pND-pet is shown. Strain BL21/pND-pet is able to utilize glucose () rapidly but barely consumed xylose (∘). In contrast, strain BL-G/pND-pet with the deletion of the ptsG gene could co-utilize both glucose (▪) and xylose (□) at a relatively slow rate. This result indicates that deletion of the ptsG gene encoding glucose permease alleviates the catabolite repression effect at the expense of the glucose transport of bacteria. FIG. 18 illustrates the ethanol production of recombinant strains. At the end of fermentation, 1.7% and 2.2% ethanol are produced by strain BL21/pND-pet (•) and BL-G/pND-pet (▪), respectively.

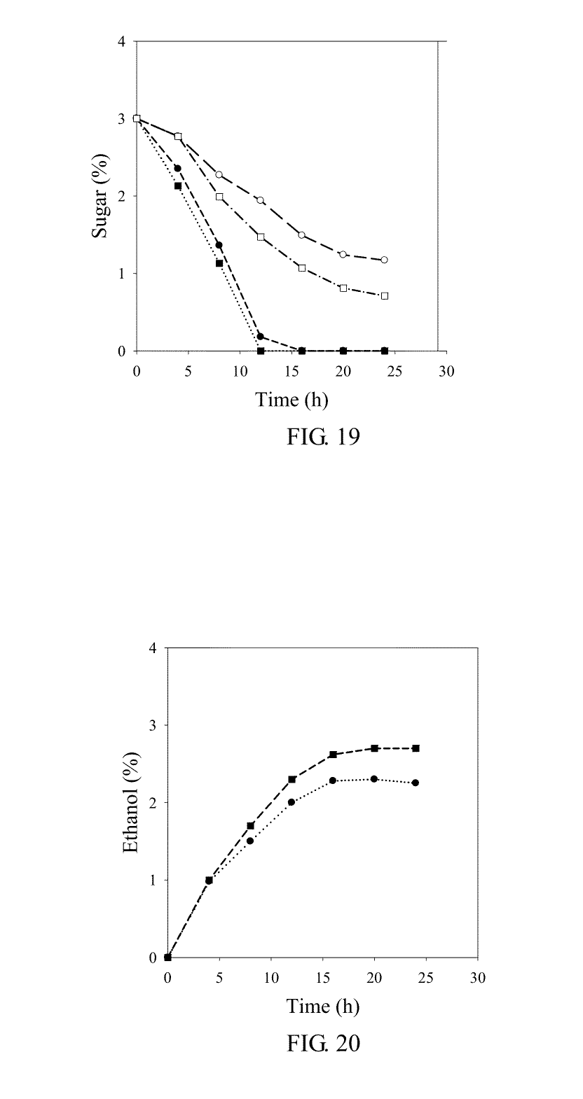

FIG. 19 illustrates the sugar consumption of the recombinant strains. The strain BL-Gf/pND-pet is isogenic to strain BL-G/pND-pet (deficient in the ptsG gene sequence) but with a genomic copy of the glf gene consumed all glucose within 14 hours. This result indicates that introduction of the glf gene encoding the glucose facilitator can resume the glucose transport ability of strain BL-G. At the end of fermentation, this strain consumed 1.8% xylose. Moreover, the rpiA, tktA, rpe, and talB gene sequences in the pentose phosphate pathway are enhanced in strain BL-Gf, thus producing strain BL21e-RB. Strain BL21e-RB/pND-pet exhibited a glucose consumption rate (▪) similar to strain BL-Gf/pND-pet (•). Nevertheless, the xylose consumption rate of strain BL21e-RB/pND-pet (□) is superior to that of strain BL-Gf/pND-pet (∘). FIG. 20 illustrates the ethanol production of strain BL-Gf/pND-pet and BL21e-RB/pND-pet. The ethanol production of the BL-Gf/pND-pet (•) reaches 2.3% and the BL21e-RB/pND-pet (▪) reaches 2.7%, respectively. This result indicates that enhanced expression of the rpiA, tktA, rpe, and talB genes can improve the xylose metabolism of the bacterium.

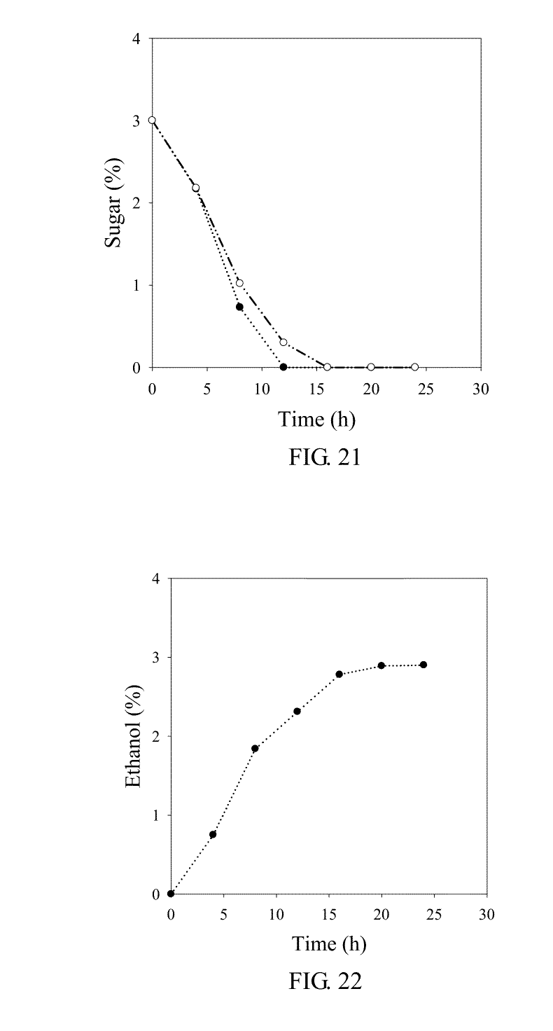

The main objective of the present invention is to construct a strain of E. coli capable of co-utilizing glucose and xylose and producing ethanol in an efficient way. For this purpose, the producer strain is constructed in a systematic manner by deletion of the ptsG gene sequence (giving strain BL-G), introduction of the glf gene sequence (giving strain BL-Gf), and enhanced expression of the rpiA, tktA, rpe, and talB genes (giving BL21e-RB). In addition, the ldhA, poxB, pta, and frdA genes of strain BL21e-RB are deleted, thus producing strain BL-A4, to curtail the waste production and to ease the inhibitory effect on the pentose phosphate pathway. In a similar culture condition, strain BL-A4/pND-pet enabled to consume both glucose and xylose simultaneously and rapidly. As shown in FIG. 21, the BL-A4/pND-pet strain metabolized all glucose (•) and xylose (∘) within 17 hours. As shown in FIG. 22, the ethanol production by BL-A4/pND-pet strain (•) can reach 2.9% at the end of fermentation. This ethanol yield accounts for 98% of the theoretical conversion yield.

Production of Lactate by Simultaneous Fermentation of Glucose and Xylose

The ability of the genetically constructed strain to co-ferment glucose and xylose for lactate production, but not limited, is illustrated within following embodiment.

Construction of Plasmid pTrc-H/D-Ldh

Forward primer 39
(SEQ ID NO: 39)
(5′-AGCTCCATGGAACTCGCCGTTTATAGCAC)
Reverse primer 40
(SEQ ID NO: 40)
(5′-AGCGAAGCTTAAACCAGTTCGTTCGGGCAG)

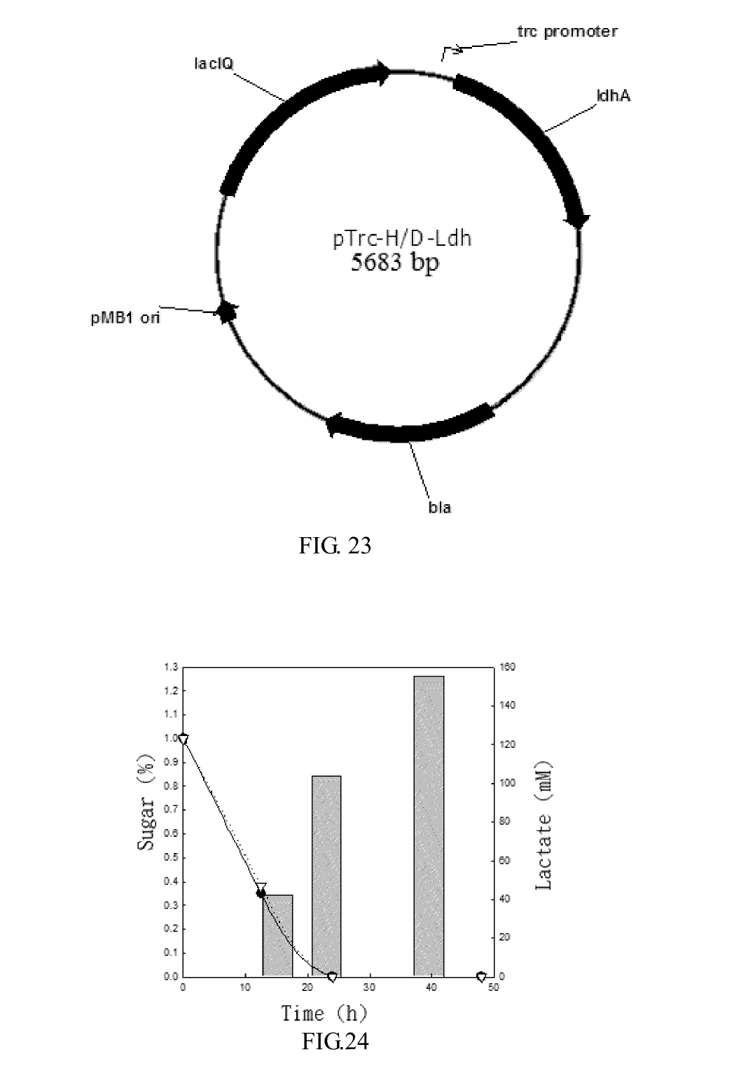

Primers 39 and 40 are synthesized based on the ldhA gene sequence in the database of NCBI; the forward primer 39 containing NcoI site (underline) and the reverse primer 40 containing the HindIII site (underline). Using the chromosome of E. coli BL21 as the template, the PCR is carried out with the primers 39 and 40. A DNA fragment (1 kb) containing the ldhA gene is amplified and purified by the Gel/PCR DNA Fragments Extraction Kit. The amplified DNA fragment is cleaved by NcoI and HindIII and recovered by Gel/PCR DNA Fragments Extraction Kit. Plasmid pTrc99A (National Institute of Genetics, Japan) purified with High-Speed Plasmid Mini kit is cleaved by NcoI and HindIII and recovered by Gel/PCR DNA Fragments Extraction Kit. The DNA fragment containing the ldhA gene and linearized plasmid pTrc99A are ligated together to obtain plasmid pTrc-H/D-Ldh from strain DH5α as shown in FIG. 23, which illustrates the anti-ampicillin gene sequence (bla), an origin of the pMB1 replication in E. coli, (pMB1 ori), a lad repressor (lacIQ), and a trc promoter (trc promoter). Plasmid pTrc-H/D-Ldh is then transformed into the BL-A4 strain to give recombinant strain BL-A4/pTrc-H/D-Ldh.

### Embodiment 3

Lactate Production by Simultaneous Fermentation of Xylose and Glucose

Another example is shown in step (i) of FIG. 2. A single colony of BL-A4/pTrc-H/D-Ldh is picked up and cultured in the LB broth (5 mL) with ampicillin at 37° C. and 200 rpm overnight. The overnight culture is seeded into 25 mL fresh LB broth with ampicillin plus 1% glucose and 1% xylose. The initial optical density (550 nm) of the culture is maintained at 0.1. The bacterial culture is then incubated at 37° C. and 200 rpm. When the optical density (550 nm) reaching 0.3, the 300 μM Isopropyl β-D-1-thiogalactopyranoside (IPTG) is added to the culture broth to induce expression of the ldhA gene sequence in strain BL-A4/pTrc-H/D-Ldh. Meanwhile, the concentration of glucose, xylose, and lactate is measured along the time course. In FIG. 24, glucose (•) and xylose (∇) are consumed simultaneously and rapidly by strain BL-A4/pTrc-H/D-Ldh. Moreover, 160 mM of lactate ( [symbol] ) is produced after 48-hour fermentation and no other organic acids are detected.

As illustrated in this embodiment, the genetically re-constructed strain BL-A4 based on the technology developed in this present invention is able to ferment glucose and xylose simultaneously and rapidly.

What is claimed is:

1. A method enabling a microorganism to ferment pentose and hexose simultaneously, which method comprises steps of:
(a) deleting a gene sequence of glucose permease in a target microorganism;
(b) introducing a glucose facilitator gene sequence into the target microorganism;
(c) introducing at least one promoter into upstream of at least one of the gene sequence in pentose phosphate pathway of the target microorganism; and
(d) deleting at least one of gene sequence responsible for synthesis of organic acid in the target microorganism.

2. The method as claimed in claim 1, wherein the target microorganism in the step (a) is Escherichia coli.

3. The method as claimed in claim 1, wherein the gene sequence of the glucose permease in step (a) is a ptsG gene sequence.

4. The method as claimed in claim 1, wherein the glucose facilitator gene sequence in the step (b) is a glf gene sequence of Zymomonas mobilis.

5. The method as claimed in claim 1, wherein the at least one of the gene sequences in the pentose phosphate pathway in the step (c) comprises a rpiA, a tktA, a rpe, a talB gene sequence or the combination thereof.

6. The method as claimed in claim 1, wherein the at least one of the gene sequences responsible for the synthesis of organic acid in the step (d) comprises a ldhA, a pta, a poxB, a frdA gene sequence or the combination thereof.

7. The method as claimed in claim 1, wherein the glucose facilitator gene sequence is introduced into the chromosome of the target microorganism in the step (b).

8. The method as claimed in claim 7, wherein the glucose facilitator gene sequence is incorporated into a plasmid, forming a first recombined plasmid; furthermore, the first recombined plasmid is transformed into the target microorganism for expression.

9. The method as claimed in claim 5, wherein the rpiA gene sequence is incorporated into a plasmid, forming a second recombined plasmid; furthermore, the second recombined plasmid is transformed into the target microorganism for expression.

10. The method as claimed in claim 5, wherein the tktA gene sequence is incorporated into a plasmid, forming a third recombined plasmid; furthermore, the third recombined plasmid is transformed into the target microorganism for expression.

11. The method as claimed in claim 5, wherein the rpe gene sequence is incorporated into a plasmid, forming a fourth recombined plasmid; furthermore, the fourth recombined plasmid is transformed into the target microorganism for expression.

12. The method as claimed in claim 5, wherein the talB gene sequence is incorporated into a plasmid, forming a fifth recombined plasmid; furthermore, the fifth recombined plasmid is transformed into the target microorganism for expression.

13. The method as claimed in claim 1, wherein a step is further comprised:
(e) introducing a gene sequence of a target product into the target microorganism; furthermore, the target microorganism be able to express the target product by fermenting the pentose and hexose simultaneously.

14. The method as claimed in claim 13, wherein the target product comprises alcohol, organic acid, disaccharide, hydrogen, ketone, alkane, or the combination thereof.

15. The method as claimed in claim 1, wherein the at least one of promoter in the step (c) is a λPRPL promoter.

16. A method enabling a microorganism to ferment pentose and hexose simultaneously comprises following steps:
(a) deleting a ptsG gene sequence in a target microorganism;
(b) introducing a glf gene sequence into the target microorganism;
(c) introducing a first promoter into upstream of a rpe and a tktA gene sequences in the target microorganism;
(d) introducing a second promoter into upstream of a rpiA and a talB gene sequences in the target microorganism;
(e) deleting a poxB gene sequence of the target microorganism;
(f) deleting a pta gene sequence of the target microorganism;
(g) deleting a ldhA gene sequence of the target microorganism; and
(h) deleting a frdA gene sequence of the target microorganism.

17. The method as claimed in claim 16, wherein the target microorganism is Escherichia coli.

18. The method as claimed in claim 16, wherein the pentose is xylose; the hexose is glucose.

19. The method as claimed in claim 16, wherein the first promoter and second promoter are λPRPL promoters in the step (c) and (d).

20. The method as claimed in claim 16, wherein the glf gene sequence in the step (b) is the glf gene sequence of Zymomonas mobilis.

21. The method as claimed in claim 16, wherein the glf gene sequence of Zymomonas mobilis is introduced into chromosome of the target microorganism.

22. The method as claimed in claim 21, wherein the glf gene sequence of Zymomonas mobilis is incorporated into a plasmid, forming a first recombined plasmid; furthermore, the first recombined plasmid is transformed into the target microorganism for expression.

23. The method as claimed in claim 16, wherein the rpiA gene sequence in the step (d) is incorporated into a plasmid, forming a second recombined plasmid; furthermore, the second recombined plasmid is transformed into the target microorganism for expression.

24. The method as claimed in claim 16, wherein the tktA gene sequence in the step (c) is incorporated into a plasmid, forming a third recombined plasmid; furthermore, the third recombined plasmid is transformed into the target microorganism for expression.

25. The method as claimed in claim 16, wherein the rpe gene sequence in the step (c) is incorporated into a plasmid, forming a fourth recombined plasmid; furthermore, the fourth recombined plasmid is transformed into the target microorganism for expression.

26. The method as claimed in claim 16, wherein the talB gene sequence in the step (d) is incorporated into a plasmid, forming a fifth recombined plasmid; furthermore, the fifth recombined plasmid is transformed into the target microorganism for expression.

27. The method as claimed in claim 16, wherein a step is further comprised:
(i) introducing a gene sequence of a target product into the target microorganism; furthermore, the target microorganism be able to express the target product by fermenting the pentose and hexose simultaneously.

28. The method as claimed in claim 27, wherein the target product in the step (i) comprises alcohol, organic acid, disaccharide, hydrogen, ketone, alkane, or the combination of thereof.


SEQUENCE LISTING (40 sequences)

SEQ ID NO: 1 - DNA, 18 bp, Escherichia coli
tgggtgaaaccgggctgg

SEQ ID NO: 2 - DNA, 18 bp, Escherichia coli
agccgtctgaccaccacg

SEQ ID NO: 3 - DNA, 20 bp, Artificial Sequence
  to comfirm an anti-kanamycin gene within the genomic ptsG gene
gattgaacaagatggattgc

SEQ ID NO: 4 - DNA, 20 bp, Artificial Sequence
  to comfirm an anti-kanamycin gene within the genomic ptsG gene
gaagaactcgtcaagaaggc

SEQ ID NO: 5 - DNA, 28 bp, Zymomonas mobilis
tgtctctagaagcatgcaggaggaatcg

SEQ ID NO: 6 - DNA, 30 bp, Zymomonas mobilis
agcaactcgagttacttctgggagcgccac

SEQ ID NO: 7 - DNA, 31 bp, Artificial Sequence
  designed based on plasmid pND-glf
aagggggatccatctaacaccgtgcgtgttg

SEQ ID NO: 8 - DNA, 30 bp, Artificial Sequence; identical to SEQ ID NO: 6
  designed based on plasmid pND-glf

SEQ ID NO: 9 - DNA, 27 bp, Escherichia coli
tatacatatgaaacagtatttgattgc

SEQ ID NO: 10 - DNA, 29 bp, Escherichia coli
cctgaattcaaacttattcatgacttacc

SEQ ID NO: 11 - DNA, 26 bp, Escherichia coli
acgggaattcaggaggagtcaaaatg

SEQ ID NO: 12 - DNA, 27 bp, Escherichia coli
gggcctcgagttacagcagttcttttc

SEQ ID NO: 13 - DNA, 31 bp, Artificial Sequence; identical to SEQ ID NO: 7
  designed based on plasmid pND-rTA

SEQ ID NO: 14 - DNA, 27 bp, Artificial Sequence
  designed based on plasmid pND-rTA
gggcctcgagttacagcagttcttttc

SEQ ID NO: 15 - DNA, 33 bp, Escherichia coli
aatgccatatgaatttcataccacaggcgaaac

SEQ ID NO: 16 - DNA, 32 bp, Escherichia coli
tggaggaattcccgtcagatcatttcacaatg

SEQ ID NO: 17 - DNA, 29 bp, Escherichia coli
tttgaattcaggaggatactatcatgacg

SEQ ID NO: 18 - DNA, 36 bp, Escherichia coli
ctaactcgaggtcgacgttacagcagatcgccgatc

SEQ ID NO: 19 - DNA, 31 bp, Artificial Sequence; identical to SEQ ID NO: 7
  designed based on plasmid pND-rTB

SEQ ID NO: 20 - DNA, 36 bp, Artificial Sequence; identical to SEQ ID NO: 18
  designed based on plasmid pND-rTB

SEQ ID NO: 21 - DNA, 31 bp, Escherichia coli
attagaagcttgcaggggtgaaacgcatctg

SEQ ID NO: 22 - DNA, 30 bp, Escherichia coli
attagactagtggctgggttgatatcaatc

SEQ ID NO: 23 - DNA, 29 bp, Escherichia coli
attaggaattcgtgattgcggtggcaatc

SEQ ID NO: 24 - DNA, 35 bp, Escherichia coli
attaggtcgacggtaccaaactggcgcaactgctg

SEQ ID NO: 25 - DNA, 30 bp, Artificial Sequence
  designed based on plasmid pKD13
ttaggaattcgtgtaggctggagctgcttc

SEQ ID NO: 26 - DNA, 20 bp, Artificial Sequence
  designed based on plasmid pKD13
attccggggatccgtcgacc

SEQ ID NO: 27 - DNA, 29 bp, Escherichia coli
tgtccaagcttattatgctgatccctacc

SEQ ID NO: 28 - DNA, 28 bp, Escherichia coli
gttcgactagtttagaaatgcgcgcgtc

SEQ ID NO: 29 - DNA, 29 bp, Escherichia coli
acgatgaattccatcagcacatctttctg

SEQ ID NO: 30 - DNA, 33 bp, Escherichia coli
accgtgtcgacggtacctgatcgcgactcgtgc

SEQ ID NO: 31 - DNA, 24 bp, Escherichia coli
tcttatgaaactcgccgtttatag

SEQ ID NO: 32 - DNA, 22 bp, Escherichia coli
ttaaaccagttcgttcgggcag

SEQ ID NO: 33 - DNA, 24 bp, Escherichia coli
gaaagtcgacgaatcccgcccagg

SEQ ID NO: 34 - DNA, 25 bp, Escherichia coli
caagaaagcttgttgataagaaagg

SEQ ID NO: 35 - DNA, 27 bp, Zymomonas mobilis
tatacatatgagttatactgtcggtac

SEQ ID NO: 36 - DNA, 32 bp, Zymomonas mobilis
ccatggatccttatcctcctccgaggagcttg

SEQ ID NO: 37 - DNA, 46 bp, Zymomonas mobilis
atgtggatccaggatatagctatggcttcttcaactttttatattc

SEQ ID NO: 38 - DNA, 31 bp, Zymomonas mobilis
aggactcgagttagaaagcgctcaggaagag

SEQ ID NO: 39 - DNA, 29 bp, Escherichia coli
agctccatggaactcgccgtttatagcac

SEQ ID NO: 40 - DNA, 30 bp, Escherichia coli
agcgaagcttaaaccagttcgttcgggcag